(19) United States
(12) Patent Application Publication
(10) Pub. No.: US 2007/0065952 A1
(43) Pub. Date: Mar. 22, 2007

(54) Multi-directional immunochromatographic assays

(76) Inventors: Paul C. Harris, Bothell, WA (US); Brian G. Richards, N. Vancouver (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/506,183

(22) Filed: Aug. 17, 2006

Related U.S. Application Data
(60) Provisional application No. 60/710,582, filed on Aug. 23, 2005.

(51) Int. Cl. G01N 33/558

(52) U.S. Cl. 436/514

(57) ABSTRACT

Methods for quantitatively measuring the amount of one or more analyte(s) of interest in a fluid sample, and kits useful in the methods, are disclosed. The methods involve providing a solid phase apparatus comprising a membrane having an application point, a sample capture zone, and a control capture zone, where the sample capture zone and the control capture zone are approximately equidistant from the application point; and providing a sample collection apparatus comprising one or more population(s) of analyte binding particles. In the assays, a fluid sample is introduced into the sample collection apparatus, and the resultant mixture is applied to the application point of the membrane. The fluid allows transport components of the assay by capillary action to and through the sample capture zone(s) and the control capture zone. The amount of each analyte of interest in the fluid sample is related (e.g., either directly or inversely) to a corrected particle amount, which can be determined, for example, as a ratio of the amount of particles in the corresponding sample capture zone and the amount of particles in the control capture zone.

### RELATED APPLICATION

This application claims the benefit of U.S. Provisional Application No. 60/710,582, filed on Aug. 23, 2005. The entire teachings of the above application are incorporated herein by reference.

### BACKGROUND OF THE INVENTION

Quantitative analysis of cells and analytes in fluid samples, particularly bodily fluid samples, often provides critical diagnostic and treatment information for physicians and patients. Quantitative immunoassays utilize the specificity of the antigen (Ag)—antibody (Ab) reaction to detect and quantitate the amount of an Ag or Ab in a sample. In solid phase immunoassays, one reagent (e.g., the Ag or Ab) is attached to a solid surface, facilitating separation of bound reagents or analytes from free reagents or analytes. The solid phase is exposed to a sample containing the analyte, which binds to its Ag or Ab; the extent of this binding is quantitated to provide a measure of the analyte concentration in the sample. Transduction of the binding event into a measurable signal, however, is affected by a number of limitations, including constraints of particle movement on the solid phase, which affect the specificity and applicability of quantitative immunoassays.

### SUMMARY OF THE INVENTION

The invention relates to methods of measuring the amount of an analyte of interest in a fluid sample, using a solid phase assay (e.g., a sandwich immunoassay or an inhibition immunoassay), in which an analyte of interest and a capture reagent are used as part of a specific binding pair; and to kits for use in the methods.

In the methods of the invention, a solid phase apparatus is provided, which includes a membrane having an application point, one or more sample capture zone(s) (one corresponding to each analyte of interest) and a control capture zone; the sample capture zone(s) and the control capture zone are approximately equidistant from the application point (e.g., parallel to one another, or radially dispersed). A sample capture reagent (e.g., an agent that binds to the analyte of interest, such as an antibody to the analyte of interest) is adsorbed in the sample capture zone for each analyte of interest. A control capture reagent (e.g., an agent that binds to the analyte binding particles, such as an anti-immunoglobulin antibody) is adsorbed in the control capture zone. Also provided is a sample collection apparatus containing a population of particles, such as liposomes, colloidal gold, or organic polymer latex particles, stored in a stable form.

In sandwich immunoassays of the invention, the particles are analyte binding particles that are coated with a binding agent (e.g., an antibody) to the analyte of interest, or are coated with a binding agent to multiple analytes of interest; alternatively, different populations of analyte binding particles, each coated with a binding agent to one of the analytes of interest, are utilized. In competitive or inhibition assays, the particles are “analyte coated” particles that are coated with analyte of interest, or are coated with multiple analytes of interest; alternatively, different populations of analyte coated particles, each coated with one of the analytes of interest, are utilized. In either type of assay, the particles can be labeled, using a colorimetric, fluorescent, luminescent, chemiluminescent, or other appropriate label, to facilitate detection.

In one embodiment of the methods, a fluid sample to be assessed for one or more analyte(s) of interest is introduced into the sample collection apparatus, and a buffer is subsequently introduced into the mixed fluid sample. In another embodiment of the methods, a buffer is introduced into the sample collection apparatus, and the fluid sample to be assessed for the analyte(s) of interest is subsequently introduced. In a third embodiment of the methods, the fluid sample is formed by introducing a solid into a buffer, and the fluid sample is subsequently introduced into the sample collection apparatus. In any of these embodiments, a buffered, mixed fluid sample containing the particles is produced.

In a sandwich assay, analyte(s) of interest present in the sample interacts with the analyte binding particles, resulting in contacted analyte binding particles within the mixed fluid sample. The buffered, mixed fluid sample is applied to the application point of the membrane of the solid phase apparatus. The solid phase apparatus is then maintained under conditions which are sufficient to allow capillary action of fluid to transport particles to and through the sample capture zone(s) and concurrently to and through the control capture zone. The sample capture reagent interacts with contacted analyte binding particles, resulting in arrest of particles in the sample capture zone(s).

Capillary action of the fluid also mobilizes the contacted analyte binding particles not only to and through the sample capture zone(s), but also concurrently to and through the control capture zone, where they bind to the control capture reagent. Capillary action of the fluid continues to mobilize the remaining unbound particles past the sample capture zone(s) and past the control capture zone (e.g., into a wicking pad). The amount of analyte binding particles that are arrested in each sample capture zone, and in the control capture zone, are then determined.

The amount of an analyte of interest in the fluid sample is then determined. For example, the amount of an analyte of interest in the fluid sample can be determined as a ratio between 1) the amount of analyte binding particles that are arrested in the sample capture zone corresponding to that analyte of interest, and 2) the amount of analyte binding particles in the control capture zone. Alternatively, the amount of an analyte of interest in the fluid sample can be determined as a ratio between 1) the amount of analyte binding particles that are arrested in the sample capture zone corresponding to that analyte of interest, and 2) the sum of the amount of analyte binding particles in the control capture zone and the amount of analyte binding particles that are arrested in the sample capture zone for that analyte of interest.

In a competitive or inhibition type of assay, the buffered, mixed fluid sample is applied to the application point of the membrane of the solid phase apparatus. The solid phase apparatus is then maintained under conditions which are sufficient to allow capillary action of fluid to transport analyte coated particles to and through the control capture zone, where they bind to the control capture reagent, and concurrently to and through the sample capture zone(s). The sample capture reagent(s) interacts with analyte coated particles; interaction of sample capture reagent(s) and analyte coated particles results in arrest of analyte coated particles in the sample capture zone(s). Because of competition between the analyte coated particles and analyte (if present) in the sample for binding sites on the sample capture reagent(s) in the sample capture zone(s), the amount of analyte coated particles arrested in the sample capture zone(s) is inversely proportional to the amount of the analyte(s) in the sample. Capillary action of the fluid continues to mobilize the remaining unbound particles past the sample capture zone(s) and the control capture zone (e.g., into a wicking pad). The amount of analyte coated particles that are arrested in the sample capture zone(s), and in the control capture zone, are then determined.

The amount of an analyte of interest in the fluid sample is then determined. For example, the amount of an analyte of interest in the fluid sample is inversely related to a ratio between 1) the amount of analyte coated particles that are arrested in the sample capture zone corresponding to that analyte of interest, and 2) the amount of analyte coated particles in the control capture zone. Alternatively, the amount of an analyte of interest in the fluid sample is inversely related to a ratio between 1) the amount of analyte coated particles that are arrested in the sample capture zone corresponding to that analyte of interest, and 2) the sum of the amount of analyte coated particles in the control capture zone and the amount of analyte coated particles that are arrested in the sample capture zone.

### BRIEF DESCRIPTION OF THE DRAWINGS

FIG. 1 depicts parallel arrangement of sample capture and/or control capture zones on a solid phase apparatus.

FIG. 2 depicts radial arrangement of sample capture and/or control capture zones on a solid phase apparatus.

### DETAILED DESCRIPTION OF THE INVENTION

The present invention pertains to methods of quantitatively measuring the amount of one or more analyte(s) of interest using assays, particularly quantitative immunochromatographic assays, and kits therefor.

An assay, as used herein, refers to an in vitro procedure for analysis of a sample to determine the presence, absence, or quantity of one or more analytes. The assays of the inventions utilize at least one analyte of interest and an analyte binding agent. Each analyte of interest and its analyte binding agent are members of a specific binding pair, in which a first member of the binding pair (e.g., analyte) reacts specifically with a second member (e.g., the binding agent). One or both members of the binding pair can be an antibody. For example, a first member of the binding pair (e.g., an analyte of interest) can be an antibody, and a second member of the binding pair (e.g., a binding agent) can be anti-immunoglobulin antibody; alternatively, the first member of the binding pair (e.g., the analyte) can be an antigen, and the second member of the binding pair (e.g., the binding agent) can be an antibody.

In one embodiment, the assay is an immunoassay which utilizes antibodies as a component of the procedure. In a preferred embodiment, the immunoassay is a sandwich assay, which is a test for an analyte in which a fluid sample to be assessed for the presence or absence, or quantity of analyte, is contacted with particles coated with an analyte binding agent, such as antibodies to the analyte, and the resultant mixture is applied to a membrane and subsequently moves by capillary action through the membrane. A positive result is indicated by detection of interaction between analyte and analyte binding agent-coated particles in a capture zone of the membrane, the amount of analyte binding agent-coated particles in the capture zone being related to the amount of analyte in the fluid sample. In another preferred embodiment, the immunoassay is an inhibition or competitive assay, which is a test for an analyte in which a fluid test sample to be assessed for the presence or absence, or quantity of analyte, is contacted with particles coated with the analyte, and the resultant mixture is applied to a membrane and subsequently moves by capillary action the system through the membrane. A positive result is indicated by detection of interaction between analyte binding agent and analyte coated particles in a capture zone of the membrane, the amount of analyte coated particles in the capture zone being inversely related to the amount of analyte in the fluid sample.

In other embodiments of the assays of the invention, neither the analyte nor the binding agent are antibodies: for example, the first member of the binding pair can be a ligand, and the second member of the binding pair can be a receptor; alternatively, the first member of the binding pair can be a lectin, and the second member of the binding pair can be a sugar. In still another embodiment, the first member of the binding pair can be a nucleic acid (e.g., DNA, RNA), and the second member of the binding pair can be a nucleic acid which specifically hybridizes to the first member of the binding pair. Specific hybridization, as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 80%, 85%, 90%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules.

Regardless of the composition of the analyte and the binding agent, these two components nevertheless form a specific binding pair, in which the first member reacts specifically with the second member. Specific interaction between the members of the binding pair indicates that the first member of the binding pair preferentially binds or otherwise interacts with the second member of the binding pair, preferably to the exclusion of any binding to another compound in the assay.

The terms, analyte or analyte of interest, as used herein, refer to a first member of a binding pair as described above. The analyte is a molecule or compound for which the amount will be measured. The analyte can be in the form of a solid, such as a dry substance (e.g., a powder, a particulate; spore; or other particle), or can be in the form of a fluid (e.g., a solid as described above that has been dissolved or suspended in a fluid; or other liquid sample). Examples of analytes include spores; proteins, such as hormones or enzymes; glycoproteins; peptides; small molecules; polysaccharides; antibodies; nucleic acids; drugs; toxins (e.g., environmental toxins); viruses or virus particles; portions of a cell wall; and other compounds. In a preferred embodiment, the analyte is “immunogenic,” which indicates that antibodies (as described below) can be raised to the analyte, or to an analyte that is bound to a carrier (e.g., a hapten-carrier conjugate, for which antibodies can be raised to the hapten). In some representative embodiments, the analyte of interest can be myoglobin; CK-MB; troponin I; PSA; digoxin; theophylline; a hormone (e.g., T-3 or T-4); a drug of abuse (LSD, THC, barbituates, etc.); or a spore of Bacillus anthracis (anthrax). The analyte of interest can be in a liquid sample; alternatively, the analyte of interest can be in a dry (non-fluid) sample (e.g., a solid, such as a particulate sample, powder sample, or soil sample). If more than one analyte of interest is being evaluated, each analyte of interest is a first member of a binding pair as described above—i.e., each analyte of interest reacts specifically with a second member of a binding pair.

In the methods of the invention, a fluid sample is assessed for the presence or absence, or quantity, of one or more analyte(s) of interest. The fluid can be a fluid that wets the membrane material; that supports a reaction between each analyte of interest and its analyte binding agent, such as the antibody/antigen reaction (i.e., does not interfere with antibody/antigen interaction); and that has a viscosity that is sufficiently low to allow movement of the fluid by capillary action. In a preferred embodiment, the fluid is an aqueous solution (such as a bodily fluid). The fluid sample can be a fluid having relatively few components, for example, an aqueous solution containing the analyte of interest; alternatively, the fluid sample can be a fluid having many components, such as a complex environmental sample (e.g., sewage, waste water, groundwater, or other water sample), or a complex biological fluid (e.g., whole blood, plasma, serum, urine, cerebrospinal fluid, saliva, semen, vitreous fluid, synovial fluid, or other biological fluid). In a preferred embodiment in which the fluid is a biological fluid, the fluid is whole blood, plasma, or serum. If desired, the fluid sample can be diluted; for example, if a complex biological fluid is used as the fluid sample, it can be diluted with a solution (e.g., an aqueous solution).

If an analyte of interest is not in solution (e.g., an analyte of interest is in a dry or solid sample, as described above), it can be extracted, suspended, or dissolved into a fluid sample first. For example, if an analyte of interest is a nucleic acid, it can be extracted from cells of interest into a solution (e.g., an aqueous solution, such as the buffer described below); in another example, if an analyte of interest is a powder or particulate material (e.g., a powder, a particulate, a soil sample, or spores), it can be suspended or dissolved into a solution (e.g., an aqueous solution, such as the buffer described below) such as by obtaining a sample of the dry material (e.g., using a swab or other instrument) and placing the sample of dry material into the solution. Thus, a fluid sample can refer not only to a liquid sample to be assessed for an analyte of interest, but also to a fluid sample in which a solid material (to be assessed for an analyte of interest) is extracted, suspended or dissolved.

An analyte binding agent, as used herein, refers to second member of a binding pair as described above. Each analyte binding agent is a compound that specifically binds to its analyte of interest (the first member of the binding pair), such as an antibody, a hapten or drug conjugate, a receptor, or another binding partner. In a preferred embodiment, an analyte binding agent is an antibody to its analyte of interest.

Sandwich Assays

The sandwich assay of the invention utilizes a solid phase apparatus. The solid phase apparatus includes a membrane having an application point, one or more sample capture zone(s), and a control capture zone. The solid phase apparatus may optionally include a wicking pad following the control capture zone, and an application pad adjacent to or covering the application point. The membrane can be made of a substance having the following characteristics: sufficient porosity to allow capillary action of fluid along its surface and through its interior; the ability to allow movement of coated particles (e.g., analyte binding particles, as described below) or complexes of particles and analyte of interest (e.g., contacted analyte binding particles, as described below) by capillary action (i.e., it must not block the particles or complexes of particles and analyte of interest); and the ability to be wet by the fluid containing the analyte (e.g., hydrophilicity for aqueous fluids, hydrophobicity for organic solvents). Hydrophobicity of a membrane can be altered to render the membrane hydrophilic for use with aqueous fluid, by processes such as those described in U.S. Pat. No. 4,340,482, or U.S. Pat. No. 4,618,533, which describe transformation of a hydrophobic surface into a hydrophilic surface. Examples of membrane substances include: cellulose, cellulose nitrate, cellulose acetate, glass fiber, nylon, polyelectrolyte ion exchange membrane, acrylic copolymer/nylon, and polyethersulfone. In a preferred embodiment, the membrane is made of cellulose nitrate (e.g., a cellulose nitrate membrane with a Mylar backing).

The application point (or application area) is the position on the membrane where a fluid can be applied. An application pad can also optionally be used; the application pad rests on the membrane, immediately adjacent to or covering the application point. The application pad can be made of an absorbent substance which can deliver a fluid sample, when applied to the pad, to the application point on the membrane. Representative substances include cellulose, cellulose nitrate, cellulose acetate, nylon, poyelectrolyte ion exchange membrane, acrylic copolymer/nylon, polyethersulfone, or glass fibers. In one embodiment, the pad is a Hemasep®-V pad (Pall Corporation). In another embodiment, the pad is a glass fiber pad. If a wicking pad is present, it can similarly be made from such absorbent substances.

A sample capture zone refers to a point on the membrane at which a sample capture reagent is adsorbed (e.g., coated on and/or permeated through the membrane). As used herein, the term “adsorbed” indicates that the agent is immobilized or adhered by non-covalent interactions, in contrast to covalent linkage where chemical means are used to generate an irreversible chemical bond of shared electrons between two linked molecules. Incremental movement of an agent that is adsorbed onto a membrane may occur, but will have negligible affect on the assays of the invention.

A sample capture reagent is an analyte binding agent, such as those described above, for a particular analyte of interest. A sample capture reagent need not be the same analyte binding agent as described in relation to analyte binding agents on particles, below; however, each sample capture reagent also forms a binding pair with its analyte of interest, in that it specifically and preferentially binds to its analyte of interest. In a preferred embodiment, the sample capture reagent is an antibody directed against its analyte of interest; it can be directed against the same epitope of the analyte as, or against a different epitope of the analyte from, the epitope that binds to the antibodies used as analyte binding agents coated on the particles. If there is more than one analyte of interest, there will accordingly be more than one sample capture zone—one sample capture zone corresponding to each analyte of interest. Each sample capture zone has a sample capture reagent adsorbed thereon, in which the sample capture reagent is an analyte binding agent for its particular (corresponding) analyte of interest.

The apparatus additionally includes a control capture reagent adsorbed in a control capture zone. The control capture reagent is a reagent which reacts with analyte binding particles, but which does not interact with any of the analytes to be measured: for example, the control capture reagent can react with analyte binding agent on analyte binding agent-coated particles; with another material on the particles; or with the particles themselves. For example, if the analyte binding agent is an antibody, the control capture reagent can be an anti-immunoglobulin antibody. In a preferred embodiment, each analyte binding agent is an antibody, and the control capture reagent is an anti-immunoglobulin antibody. The control capture reagent is adsorbed on the membrane (coated on and/or permeated in the membrane) in a control capture zone.

The control capture zone and the sample capture zone(s) are positioned such that they are approximately equidistant from the application point. For example, in a representative embodiment of the invention, if the application point is approximately 5 mm in length and a capture zone are approximately 1-2 mm in length, the distance from the application point to the sample capture zone can be approximately 20-50 mm, preferably 25-40 mm, even more preferably 30-40 mm, and the distance from the center of the application point to the center of a sample capture zone and the distance from the center of the application point to the center of the control capture zone are approximately equidistant. In one embodiment, they vary by 5 mm or less, and even more preferably by 1 mm or less: for example, if the distance from the center of the application point to the center of the control capture zone is 35 mm, the distance from the center of the application point to the center of each sample capture zone will be within 5 mm of 35 mm—that is, from 30 to 40 mm, and more preferably within 1 mm of 35 mm—that is, from 34 to 36 mm. In a particular embodiment, “approximately equidistant” indicates that the distance is as close as possible using standard manufacturing equipment: for example, if the manufacturing equipment resolution is a millimeter, approximately equidistant would be within 1 mm. Alternatively, in another particular embodiment, approximately equidistant resolution can be related to the distance from the center of the application point to the center of a sample capture zone (the length of the pathway): for example, the difference between the distance from the center of the application point to the center of a sample capture zone and the distance from the center of the application point to the center of the control capture zone, is within 10%, preferably within 7%, preferably within 5%, more preferably within 4%, more preferably within 3%, even more preferably within 2%, and even more preferably within 1%, of the length of the distance from the center of the application point to the center of a sample capture zone (the length of the pathway).

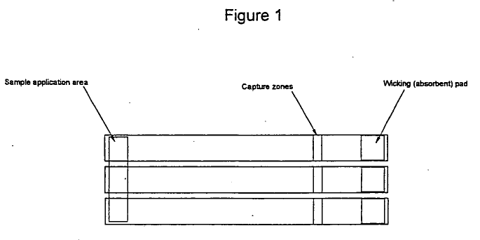

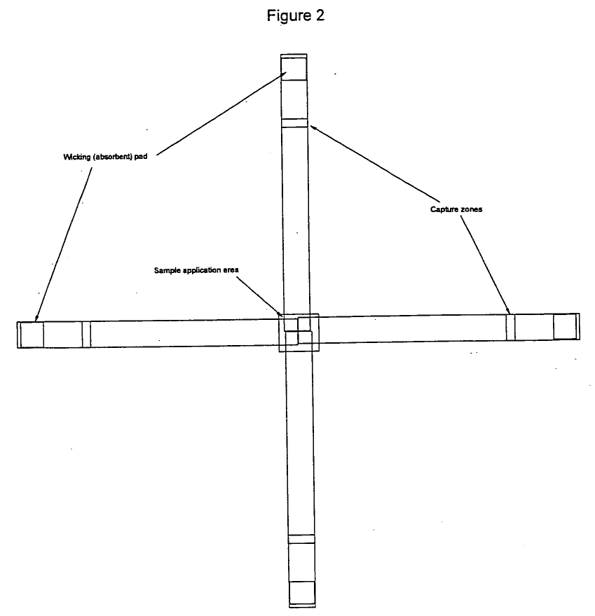

In certain embodiments, the control capture zone and the sample capture zone(s) are radially dispersed around the application point (see, for example, FIG. 2); in other embodiments, the control capture zone and the sample capture zone(s) are adjacent to one another and situated in parallel along the direction of fluid flow by capillary action (see, for example, FIG. 1). Thus, each sample capture zone and the control capture zone are approximately equally distant from a central point (e.g., in the case of a radial arrangement), or are approximately equally distant (e.g., in the case of a parallel arrangement). It should be noted that the capillary paths from the application point to each sample capture zone(s) and to the control capture zone do not cross: that is, the path of fluid flow from the application point to each capture zone remains distinct and does not cross over any other path of fluid flow.

Furthermore, in a preferred embodiment, the sample capture zone(s) and the control capture zone are separated from the application point by a space that is sufficiently large to retard the speed of the capillary front to a rate that is slow enough to allow capture of particles when the capillary front reaches the sample capture zone. In addition, the distance must be sufficiently large so that the total time of migration (movement of the capillary front through the entire membrane) is long enough to allow free analyte in a fluid sample to bind to analyte binding particles. The optimal distances between the components on the membrane can be determined and adjusted using routine experimentation.

The quantitative assay additionally uses a sample collection apparatus. A sample collection apparatus, as used herein, refers to an apparatus that can be used for collection of the fluid sample or into which a collected fluid sample can be deposited or stored. The sample collection apparatus can be any apparatus which can contain the analyte binding particles, as described below, and which to which can be added a measured volume of fluid sample. Representative sample collection apparatus include a sample tube, a test tube, a vial, a pipette or pipette tip, or a syringe. In a preferred embodiment, the sample collection apparatus is a pipette or pipette tip.

The sample collection apparatus contains a population of analyte binding particles which are coated with an analyte binding agent, in the case of a single analyte of interest. If there is more than one analyte of interest, the analyte binding particles are coated with an analyte binding agent for each analyte of interest: for example, a first analyte binding agent for a first analyte of interest; a second analyte binding agent for a second analyte of interest; etc., such that there is an analyte binding agent corresponding to each analyte of interest. Alternatively, the sample collection apparatus can contain a population of analyte binding particles for each analyte binding agent that is, a population of analyte binding particles for a first analyte of interest; a population of analyte binding particles for a second analyte of interest; etc., such that there is a population of analyte binding particles corresponding to each analyte of interest. The population of particles varies, depending on the size and composition of the particles, the composition of the membrane of the solid phase apparatus, and the level of sensitivity of the assay. The population typically ranges approximately between 1×10³ and 1×10⁹, although fewer or more can be used if desired. In a preferred embodiment, the population is approximately 2×10⁸ particles. If more than one analyte of interest is assessed, the population may be accordingly increased if desired (e.g., with three times as many particles if three analytes of interest are assessed). Analyte binding particles are particles which can be coated with the analyte binding agent (the second member of the binding pair) for each analyte of interest. In a preferred embodiment, the analyte binding particles are liposomes, colloidal gold, organic polymer latex particles, inorganic fluorescent particles or phosphorescent particles. In a particularly preferred embodiment, the particles are polystyrene latex beads, and most particularly, polystyrene latex beads that have been prepared in the absence of surfactant, such as surfactant free Superactive Uniform Aldehyde/Sulfate Latexes (Interfacial Dynamics Corp., Portland, Oreg.).

The size of the particles is related to porosity of the membrane (for analytes in fluid samples) and also to the size of the analyte(s) of interest (e.g., for particulate analytes): the particles must be sufficiently small to be transported along the membrane by capillary action of fluid, and also (for solid, e.g., particulate analytes,) sufficiently small for the complex of contacted analyte binding particles, as described below, to be transported along the membrane by capillary action. The particles can be labeled to facilitate detection. The particles are labeled by a means which does not significantly affect the physical properties of the particles; for example, the particles are labeled internally (that is, the label is included within the particle, such as within the liposome or inside the polystyrene latex bead). Representative labels include luminescent labels; chemiluminescent labels; phosphorescent labels; enzyme-linked labels; chemical labels, such as electroactive agents (e.g., ferrocyanide); and colorimetric labels, such as dyes or fluorescent labels. In one embodiment, a fluorescent label is used. In another embodiment, phosphorescent particles are used, particularly “up-converting” phosphorescent particles, such as those described in U.S. Pat. No. 5,043,265.

The particles are coated with an analyte binding agent that is a second member of the binding pair for each analyte of interest (e.g., particles having more than one type of analyte binding agent coated thereon; or different populations of particles, each population having a single type of analyte binding agent for its analyte coated thereon). As described above, an analyte binding agent (second member of a binding pair) specifically and preferentially binds to its analyte of interest (first member of the binding pair). Representative analyte binding agents include antibodies (or fragments thereof); haptens; drug conjugates; receptors; or other binding partners. In one preferred embodiment, the analyte binding agent is an antibody to the analyte of interest. Antibodies can be monoclonal antibodies or polyclonal antibodies. The term “antibody”, as used herein, also refers to antibody fragments which are sufficient to bind to the analyte of interest. Alternatively, in another embodiment, molecules which specifically bind to the analyte of interest, such as engineered proteins having analyte binding sites, can also be used (Holliger, P. and H. R. Hoogenbloom, Trends in Biotechnology 13:7 9 (1995); Chamow, S. M. and A. Ashkenazi, Trends in Biotechnology 14:52 60:1996)). In still another embodiment, if the analyte of interest is a drug, a hapten or other drug conjugate can be used as the analyte binding agent. Alternatively, in a further embodiment, a receptor which binds to the analyte can be used (e.g., if the analyte of interest is a ligand). If the analyte is an antibody of known specificity, the particles can be coated with the antigen against which the analyte antibody is directed, or can be coated with antibody to the analyte-antibody. Furthermore, because the analyte and the analyte binding agent form a binding pair, compounds or molecules described as representative analytes can also serve as analyte binding agents, and those described as representative analyte binding agents can similarly serve as analytes, as described herein.

The analyte binding particles contained within the sample collection apparatus are stored in a stable form within the sample collection apparatus. A “stable form,” as the term is used herein, indicates a form in which the particles do not significantly change in chemical makeup or physical state during storage. The stable form can be a liquid, gel, or solid form. In preferred embodiments, the analyte binding particles contained within the sample collection apparatus are evaporatively dried; freeze-dried; and/or vacuum-dried.

In a particularly preferred embodiment, the sample collection apparatus is a pipette tip in which are vacuum-dried analyte binding particles.

To perform the assay, a fluid sample to be assessed for the presence of the analyte(s) of interest, as described above, is used. In one embodiment, the fluid sample is introduced into (drawn into, poured into, or otherwise placed into) the sample collection apparatus. For example, in one embodiment, the fluid sample is drawn up into a sample collection apparatus that comprises a pipette tip. Introduction of the fluid sample into the sample collection apparatus results in mixing of the fluid sample with the analyte binding particles, forming a “mixed fluid sample.” If the analyte binding particles are evaporatively-, freeze- or vacuum-dried, the introduction of the fluid sample into the sample collection apparatus can result in rehydration and suspension of the analyte binding particles in the fluid sample. A buffer (e.g, for dilution) is also introduced into the mixed fluid sample, forming a “buffered, mixed fluid sample.” The buffered, mixed fluid sample can be formed either by dispensing the mixed fluid sample into a “buffer container” (e.g., test tube) containing the buffer, or by introducing the buffer into the sample collection apparatus prior to introducing the fluid sample. Alternatively, if the analyte of interest is a solid (e.g., a powder, a particulate; spore; or other particle, as described above), the fluid sample as described above can be prepared by introducing the solid into the buffer container; in this embodiment, the buffered, mixed fluid sample is formed by introducing the fluid sample (comprising the buffer) into the sample collection apparatus. In another embodiment, the buffer is introduced into the sample collection apparatus, followed by introduction of the fluid sample into the sample collection apparatus.

The buffer can be an aqueous fluid that supports a reaction between the analyte of interest and the analyte binding agent (e.g., does not interfere with antibody/antigen interaction); and that has a viscosity that is sufficiently low to allow movement of the fluid by capillary action. In one embodiment, the buffer contains one or more of the following components: a buffering agent (e.g., phosphate); a salt (e.g., NaCl); a protein stabilizer (e.g., BSA, casein, serum); and/or a detergent such as a nonionic detergent or a surfactant (e.g., one or more of the following agents commonly available in surfactant tool kits: NINATE 411, Zonyl FSN 100, Aerosol OT 100%, GEROPON T 77, BIO TERGE AS 40, STANDAPOL ES 1, Tetronic 1307, Surfnyol 465, Surfynol 485, Surfynol 104PG 50, IGEPAL CA210, TRITON X 45, TRITON X 100, TRITON X305, SILWET L7600, RHODASURF ON 870, Cremophor EL, TWEEN 20, TWEEN 80, BRIJ 35, CHEMAL LA 9, Pluronic L64, SURFACTANT 10G, SPAN 60, CREL). Optionally, if desired, the buffer can contain a thickening agent. Such components for buffers are commercially available. Representative buffers include, for example, saline, or 50 mM Tris HCl, pH 7.2. Alternatively, water can be used in lieu of a buffered solution; as used herein, the term “buffer” refers to either a buffered solution or to water.

To disperse the analyte binding particles further into the fluid sample, if desired, the sample collection apparatus into which the fluid sample and the buffer has been introduced, or the buffer container into which the mixed fluid sample has been introduced, can be agitated (e.g., vortexed, shaken, pipetted down and up, etc.).

In a preferred embodiment, the sample collection apparatus comprises a pipette tip having vacuum-dried analyte binding particles within its tip; the fluid sample is drawn into the pipette, thereby rehydrating the dried analyte binding particles and forming a mixed fluid sample. In a particularly preferred embodiment, the mixed fluid sample is introduced into a buffer container, resulting in a buffered mixed fluid sample; the buffered mixed fluid sample in the buffer container is pipetted up and down using the sample collection apparatus, thereby further dispersing the analyte binding particles.

If an analyte of interest is present in the buffered, mixed fluid sample, binding occurs between that analyte and its analyte binding particles. “Binding” of analyte to analyte binding particles indicates that an analyte binding agent coated onto the particle is interacting with (e.g., binding to) its analyte of interest. Analyte binding particles which have been maintained (incubated) under conditions allowing analyte(s) in the fluid (if present) to bind to analyte binding particles adsorbed in the contact region are referred to herein as “contacted analyte binding particles”. Contacted analyte binding particles may or may not have analyte(s) bound to the analyte binding agent, depending on whether or not each analyte of interest is present in the fluid sample and whether analyte has bound to the analyte binding agent on the analyte binding particles. Because there are multiple binding sites for analyte on analyte binding particles, the presence and the concentration of analyte bound to analyte binding particles varies; the concentration of analyte bound to the analyte binding particles increases proportionally with the amount of analyte present in the fluid sample, and the probability of an analyte binding particle being arrested in the sample capture zone (as described below) similarly increases with increasing amount of analyte bound to the analyte binding particles. Thus, the population of contacted analyte binding particles may comprise particles having various amount of analyte bound to the analyte binding agent, as well as particles having no analyte bound to the analyte binding agent (just as the analyte binding particles initially have no analyte bound to the analyte binding agent). Furthermore, the degree of binding increases as the time factor of the conditions increases: while the majority of binding occurs within one minute (e.g., 60 seconds, preferably less than 60 seconds (e.g., 45 seconds, 30 seconds, or less), additional incubation (e.g., more than one minute (2 minutes, 5 minutes, 10 minutes, 15 minutes) results in additional binding. If there is more than one population of analyte binding particles (e.g., separate populations for different analytes of interest), analyte binding particles which have been maintained (incubated) under conditions allowing analyte(s) in the fluid (if present) to bind to the analyte binding particles are referred to as “contacted first analyte binding particles,” “contacted second analyte binding particles,” etc., and are collectively known as contacted analyte binding particles.

The buffered, mixed fluid sample is applied to the application point of the membrane of the solid phase apparatus, or to the application pad, if present. After the membrane is contacted with the buffered, mixed fluid sample, the membrane is maintained under conditions which allow fluid to move by capillary action to and through the membrane. Contacted analyte binding particles move through the membrane as a result of capillary action of the fluid from the buffered, mixed fluid sample, and the contacted analyte binding particles move along the membrane to and through the sample capture zone(s) on the membrane as well as to and through the control capture zone. The membrane is maintained under conditions (e.g., sufficient time and fluid volume) which allow contacted analyte binding particles to move by capillary action along the membrane to and through the sample capture zone(s) and to and through the control capture zone, and subsequently beyond the capture zones (e.g., into a wicking pad), thereby removing any non-bound particles from the capture zones.

The movement of some of the contacted analyte binding particles is arrested by binding of contacted analyte binding particles to the sample capture reagent in the sample capture zone for each analyte of interest, and concurrently by binding of some of the contacted analyte binding particles to the control capture reagent in the control capture zone. In one preferred embodiment, the analyte binding agent(s) is antibody to the antigen of interest, and the control capture reagent can be antibody against immunoglobulin of the species from which the analyte binding agent is derived. In this embodiment, the antibody to immunoglobulin should be non-cross reactive with other components of the sample: for example, if a human sample is being tested, an antibody that does not react with human immunoglobulin can be used as the control capture reagent.

Sample capture reagent binds to contacted analyte binding particles by binding to analyte of interest which is bound to analyte binding agent on the contacted analyte binding particles. The term, sample-reagent particle complexes, as used herein, refers to a complex of sample capture reagent and contacted analyte binding particles. Contacted analyte binding particles are arrested in the sample capture zone, forming the sample-reagent-particle complexes, due to capture of contacted analyte binding particles by interaction of analyte with sample capture reagent in the sample capture zone. Each sample capture zone may have sample-reagent-particle complexes arrested therein, depending on whether each particular analyte of interest is present in the sample and has bound to its analyte binding agent on contacted analyte binding particles.

Control capture reagent binds to contacted analyte binding particles by binding to analyte binding agent on the contacted analyte binding particles. The term, control-reagent-particle complexes, as used herein, refers to a complex of the control capture reagent and contacted analyte binding particles. Contacted analyte binding particles are arrested in the control capture zone, forming the control-reagent-particle complexes, due to capture of contacted analyte binding particles by interaction of analyte binding particles with control capture reagent in the control capture zone. As indicated above, the control capture reagent interacts with analyte binding particles (e.g., with the analyte binding agent on the analyte binding agent-coated particles, or another material on the particles, or with the particles themselves), but not with the analyte itself.

Capillary action subsequently moves any contacted analyte binding particles that have not been arrested in either a sample capture zone or the control capture zone, onwards beyond these zones, thereby removing any particles that have not been arrested. In a preferred embodiment, the fluid moves any contacted analyte binding particles that have not been arrested, into a wicking pad which follows each sample capture zone and the control capture zone.

If desired, a secondary wash step can be used. A buffer (e.g., the buffer described above) can be applied at the application point after the buffered, mixed fluid sample has soaked in to the membrane or into the application pad, if present. The secondary wash step can be used at any time thereafter, provided that it does not dilute the buffered, mixed fluid sample. A secondary wash step can contribute to reduction of background signal when the analyte binding particles are detected, as described below.

The amount of analyte binding particles arrested in each sample capture zone (sample-reagent-particle complexes) is then detected using an appropriate means for the type of label used on the analyte binding particles. In a preferred embodiment, the amount is detected by an optical method, such as by measuring the amount of fluorescence of the label of the analyte binding particles. Alternatively, the amount of sample-reagent-particle complexes can be detected using electrical conductivity or dielectric (capacitance). Alternatively, electrochemical detection of released electroactive agents, such as indium, bismuth, gallium or tellurium ions, as described by Hayes et al. (Analytical Chem. 66:1860-1865 (1994)) or ferrocyanide as suggested by Roberts and Durst (Analytical Chem. 67:482-491 (1995)) can be used. For example, if liposomes are used, ferrocyanide encapsulated within the liposome can be released by addition of a drop of detergent at the capture zone, and the released ferrocyanide detected electrochemically (Roberts and Durst, id.). If chelating agent-protein conjugates are used to chelate metal ions, addition of a drop of acid at the capture zone will release the ions and allow quantitation by anodic stripping voltametry (Hayes et al., id.). Similarly, the amount of analyte binding particles arrested in the control capture zone is detected in the same manner as the amount of analyte binding particles in a sample capture zone.

In one embodiment, the detected amount of analyte binding particles is represented by a curve that is directly related to the amount of label present at positions along the solid phase (e.g., the membrane). For example, the detected amounts of particles at each position on the membrane (e.g., at the sample capture zone and the control capture zone, and/or areas in between or adjacent to the sample capture zone and the control capture zone, and/or other areas of the membrane) can be determined and plotted as a function of the distance of the position along the membrane. The amount of particles can then be calculated as a function of the area under the curve, which is related to the amount of label present.

A corrected analyte binding particle amount is then determined, and the amount of an analyte of interest can then be determined from the corrected analyte binding particle amount for that analyte using appropriate calculation. A corrected analyte binding particle amount is based on the amount of analyte binding particles arrested in the sample capture zone corresponding to analyte of interest, and in the control capture zone. For example, in one embodiment, the corrected analyte binding particle amount is determined as a ratio (R) of the analyte binding particle amount present in the sample capture zone to the analyte binding particle amount present in the control capture zone. The amount of analyte present can be then determined from the corrected analyte binding particle amount (the ratio), utilizing a standard curve. The standard curve is generated by preparing a series of control samples, containing known concentrations of the analyte of interest in the fluid in which the analyte is to be detected (for example, such as serum depleted of the analyte). The assay is then performed on the series of control samples; the value of R is measured for each control sample; and the R values are plotted as a function of the concentration of analyte included in the control sample. Samples containing an unknown amount of analyte (the “test samples”) are assayed by measuring the value of R for the test sample, and the concentration of analyte in the test sample is determined by referring to the standard curve. As above, one standard curve can be generated and used for all test samples in a lot (e.g., for all test samples using a specified preparation of test reagents); it is not necessary that the standard curve be re generated for each test sample. In another embodiment, the corrected analyte binding particle amount is determined as a ratio (R) of the amount of the analyte binding particle amount present in the sample capture zone, to the sum of the analyte binding particle amount present in the control capture zone and the analyte binding particle amount present in the sample capture zone. The amount of analyte present can be then determined from corrected analyte binding particle amount (the ratio), utilizing a standard curve. Alternatively, other ratios and/or standard curves can also be used to determine the amount of analyte in the sample. In addition, if desired, the amount of label that is present in the background can be subtracted from the analyte binding particle amount present in the sample capture zone and the analyte binding particle amount present in the control capture zone prior to calculation of the ratio (R).

For example, after the assay is run (liquid has moved through and beyond the capture zones), the whole, or part, of the membrane can be scanned to assess the quantity of labeled particles in the areas before, in, and after the capture zones. The scan can be done primarily around the area which includes the capture zone(s), but can also be performed on the area extending outside of these zones. The particles present in areas outside the capture zone(s) are “background”—that is, particles that bind non-specifically to the membrane in the presence of the sample and other constituents in the sample matrix which are also present at the capture zone(s). The amount of particles present in the capture zone includes this non-specific background in addition to the specific particles captured by the capture reagent. The background amount of particles (taken before and/or after the capture zone) can be subtracted from the total amount of particles determined in the capture zone(s). This yields a corrected background amount, which can yield more accurate determination of the amount of analyte present in the sample.

If more than one analyte of interest is examined, the corrected analyte binding particle amount is determined individually for each analyte of interest, using the amount of analyte binding particles arrested in the sample capture zone corresponding to that analyte of interest, and the amount of analyte binding particles arrested in the control capture zone. Thus, the amount of analyte binding particles in the control capture zone will be used to determine the corrected analyte binding particle amount for all of the analytes of interest, even though the amount of analyte binding particles in each individual sample capture zone will be used only for determination of the corrected analyte binding particle amount for that particular analyte of interest.

It should be noted that the methods described above for the assessment of multiple analytes of interest, can also be applied to analysis of multiple analytes of interest in which the analytes of interest are all the same (i.e., a single analyte assessed multiple times). In this embodiment, the same sample capture reagent can be used in each of the sample capture zones. If desired, the corrected analyte binding particle amounts can be averaged, and the amount of analyte of interest will be related to the resultant average corrected analyte binding particle amount.

“Competitive” or “Inhabition” Assays

The competitive or inhibition assay of the invention, like the sandwich assays, utilizes a solid phase apparatus including a membrane, as described above, that includes an application point, one or more sample capture zone(s), and a control capture zone. The membrane may optionally include a wicking pad following the control capture zone and/or the sample capture zone(s), and a sample pad preceding the application point. This embodiment also utilizes a sample collection apparatus, as described above. In certain embodiments, the sample collection apparatus for the competitive (inhibition) assay contains a population of analyte coated particles which are coated with the analyte of interest (in lieu of being coated with an analyte binding agent, as described for the sandwich assays) or with an analog of the analyte of interest. In other embodiments in which there is more than one analyte of interest, the analyte coated particles are coated with all of the different analytes of interest (or with analogs of the analytes of interest, or with a combination of one analyte of interest and analog of another analyte of interest, etc.); alternatively, the sample collection apparatus contains more than one population of analyte coated particles (with one population for each analyte of interest); each population is coated with an analyte of interest or with an analog of an analyte of interest. An analog of the analyte, as used herein, is a compound that has similar binding characteristics as the analyte, in that is forms a binding pair with the analyte-binding agent as described above. The analyte or analog of the analyte can be coated directly on the particles, or can be indirectly bound to the particles. As used below, the term analyte coated particles can refer to particles that are coated either with an analyte of interest or with an analog of an analyte of interest. As above with regard to the sandwich assay, the population of particles varies, depending on the size and composition of the particles, the composition of the membrane of the solid phase apparatus, and the level of sensitivity of the assay.

As above, the sample capture zone(s) refers to a point on the membrane at which a sample capture reagent is adsorbed. The sample capture reagent is an analyte binding agent, such as those described above. The sample capture reagent need not be the same analyte binding agent as described above; however, the sample capture reagent also forms a binding pair with the analyte of interest, in that it specifically and preferentially binds to an analyte of interest. If there is more than one analyte of interest, there will be more than one sample capture zone, as above. As above, in a preferred embodiment, the sample capture reagent is an antibody directed against the analyte; it can be directed against the same epitope of the analyte as, or against a different epitope of the analyte from, the epitope that binds to the antibodies used as analyte binding agents coated on the particles.

The apparatus additionally includes a control capture reagent, as described above, that reacts with the analyte coated particles, but does not interact with the analyte to be measured: for example, the control capture reagent can react with another material on the particles (e.g., a carrier for the analyte that is bound to the particles; an antibody); or with the particles themselves. In a preferred embodiment, the sample capture reagent and the control capture agent are both antibodies. The control capture reagent is adsorbed on the membrane (coated on and/or permeated in the membrane) in the control capture zone. The components of the competitive assay are positioned in a similar manner as described above with regard to the sandwich assay.

To perform the competitive assay, a fluid sample to be assessed for the presence of the analyte of interest, as described above, is used. In one embodiment, the fluid sample is introduced into (drawn into, poured into, or otherwise placed into) the sample collection apparatus. For example, in one embodiment, the fluid sample is drawn up into a sample collection apparatus that comprises a pipette tip. Introduction of the fluid sample into the sample collection apparatus results in mixing of the fluid sample with the analyte coated particles, forming a mixed fluid sample. If the analyte coated particles are evaporatively-, freeze- or vacuum-dried, the introduction of the fluid sample into the sample collection apparatus can result in rehydration and suspension of the analyte binding particles in the fluid sample. A buffer (e.g., as described above) is also introduced into the mixed fluid sample, forming a buffered, mixed fluid sample. The buffered, mixed fluid sample can be formed either by dispensing the mixed fluid sample into a buffer container (e.g., test tube) containing the buffer, or by introducing the buffer into the sample collection apparatus prior to introducing the fluid sample. In another embodiment, the buffer is introduced into the sample collection apparatus, followed by introduction of the fluid sample into the sample collection apparatus. Alternatively, if analyte of interest is a solid (e.g., a powder, a particulate; spore; or other particle, as described above), the fluid sample as described above can be prepared by introducing the solid into the buffer container; in this embodiment, the buffered, mixed fluid sample is formed by introducing the fluid sample (comprising the buffer) into the sample collection apparatus.

To disperse the analyte coated particles further into the fluid sample, if desired, the sample collection apparatus into which the fluid sample and the buffer has been introduced, or the buffer container into which the mixed fluid sample has been introduced, can be agitated (e.g., vortexed, shaken, pipetted down and up, etc.).

In a preferred embodiment, the sample collection apparatus comprises a pipette tip having vacuum-dried analyte coated particles within its tip; the fluid sample is drawn into the pipette, thereby rehydrating the dried analyte coated particles and forming a mixed fluid sample. In a particularly preferred embodiment, the mixed fluid sample is introduced into a buffer container, resulting in a buffered mixed fluid sample; the buffered mixed fluid sample in the buffer container is pipetted up and down using the sample collection apparatus, thereby further dispersing the analyte coated particles.

The buffered, mixed fluid sample is applied to the application point of the membrane of the solid phase apparatus, or to the application pad, if present. After the membrane is contacted with the buffered, mixed fluid sample, the membrane is maintained under conditions which allow fluid to move by capillary action to and through the membrane. The analyte coated particles (and analyte, if present in the sample) move through the membrane as a result of capillary action of the fluid from the buffered, mixed fluid sample, to and through the sample capture zone(s) on the membrane and concurrently to and through the control capture zone. The membrane is maintained under conditions (e.g., sufficient time and fluid volume) which allow the analyte coated particles to move by capillary action along the membrane to and through the sample capture zone and concurrently to the control capture zone, and subsequently beyond the capture zones (e.g., into a wicking pad), thereby removing any non-bound particles from the capture zones.

The movement of some of the analyte coated particles is arrested by binding of analyte coated particles to the sample capture reagent in the sample capture zone(s), and also by binding of some of the analyte coated particles to the control capture reagent in the control capture zone. The analyte coated particles compete with analyte (if present) in the sample for binding to the sample capture reagent. The sample capture reagent binds to analyte coated particles by binding to analyte on the analyte coated particles. The term, sample-reagent-analyte coated particle complexes, as used herein, refers to a complex of the sample capture reagent and analyte coated particles. The analyte coated particles are arrested in a sample capture zone, forming the sample-reagent-analyte coated-particle complexes, due to capture of the analyte coated particles by interaction of the analyte of interest on the particles with the sample capture reagent in the sample capture zone.

The control capture reagent binds to analyte coated particles by binding to any component of the analyte coated particles except the analyte itself. The term, control-reagent-analyte coated particle complexes, as used above, refers to a complex of the control capture reagent and analyte coated particles. As above, the analyte coated particles are arrested in the control capture zone, forming the control-reagent-analyte coated particle complexes, due to capture of the analyte coated particles by interaction of the analyte binding particles with the control capture reagent in the control capture zone.

Capillary action subsequently moves any analyte coated particles that have not been arrested in either a sample capture zone or the control capture zone, onwards beyond the capture zones. In a preferred embodiment, the fluid moves any contacted analyte coated particles that have not been arrested in either capture zone into a wicking pad which follows the control capture zone.

The amount of analyte coated particles arrested in each sample capture zone is then detected. The analyte coated particles are detected using an appropriate means for the type of label used on the analyte coated particles. In a preferred embodiment, the amount of analyte coated particles is detected by an optical method, such as by measuring the amount of fluorescence of the label of the analyte-binding particles. The amount of analyte coated particles arrested in the control capture zone is detected in the same manner as the amount of analyte coated particles in the sample capture zone(s). In one embodiment, as described above, the amount of analyte coated particles is represented by a curve that is directly related to the amount of label present at positions along the solid phase (e.g., the membrane). For example, the amount of particles at each position on the membrane (e.g., at the sample capture zone and the control capture zone, and/or areas in between or adjacent to the sample capture zone and the control capture zone, and/or other areas of the membrane) can be determined and plotted as a function of the distance of the position along the membrane. The amount of particles can then be calculated as a function of the area under the curve, which is related to the amount of label present.

A corrected analyte coated particle amount is determined, and the amount of analyte can then be determined from the corrected analyte coated particle amount using appropriate calculation. The corrected analyte coated particle amount is based on the amount of analyte coated particles arrested in a sample capture zone and in the control capture zone. For example, in one embodiment, the corrected analyte coated particle amount is inversely proportional to a ratio (R) of an analyte coated particle amount present in a sample capture zone to the analyte coated particle amount present in the control capture zone. The amount of analyte present can be then determined from the corrected analyte coated particle amount (the ratio), utilizing a standard curve. The standard curve is generated by preparing a series of control samples, containing known concentrations of the analyte of interest in the fluid in which the analyte is to be detected (such as serum depleted of the analyte). The assay cam then performed on the series of control samples; the value of R is measured for each control sample; and the R values are plotted as a function of the concentration of analyte included in the control sample. Samples containing an unknown amount of analyte (the “test samples”) are assayed by measuring the value of R for the test sample, and the concentration of analyte in the test sample is determined by referring to the standard curve. As above, one standard curve can be generated and used for all test samples in a lot (e.g., for all test samples using a specified preparation of test reagents); it is not necessary that the standard curve be re generated for each test sample. In another embodiment, the corrected analyte coated particle amount is inversely proportional to a ratio (R) of the amount of the analyte coated particle amount present in the sample capture zone, to the sum of the analyte coated particle amount present in the control capture zone and the analyte coated particle amount present in the sample capture zone. The amount of analyte present can be then determined from corrected analyte coated particle amount (the ratio), utilizing a standard curve. Alternatively, other ratios and/or standard curves can also be used to determine the amount of analyte in the sample. In addition, if desired, the amount of label that is present in the background can be subtracted from the analyte coated particle amount present in the sample capture zone and the analyte coated particle amount present in the control capture zone prior to calculation of the ratio (R), as described previously in relation to sandwich assays.

If there is more than one analyte of interest, each corrected analyte coated particle amount is determined individually for each analyte of interest, using the amount of analyte coated particles arrested in a sample capture zone for each analyte of interest.

### BENEFITS OF THE INVENTION

The methods of the invention provide assays with enhanced sensitivity, when compared with assays in which the analyte binding particles are imbedded within the membrane of the solid phase apparatus. For the sandwich assays, for example, because the fluid sample to be assayed for the analyte of interest is mixed with the analyte binding particles prior to application to the membrane, there is a longer time for the analyte of interest to bind to the analyte binding particles prior to the capture reaction which occurs in the membrane. Furthermore, because the interaction between the analyte of interest and the analyte binding particles occurs in the fluid phase, it allows more efficient binding because of greater mobility of the particles, than the same interaction between analyte of interest and analyte binding particles would be in the matrix of the membrane of the solid phase apparatus. Also, with regard to both the sandwich and the competitive assays, a greater number of particles can be included in a fluid collection apparatus than would be possible to embed in a solid phase apparatus; the greater number further enhances the sensitivity of the reaction. In addition, because the analyte binding particles (or analyte coated particles) are dispersed in the buffered, mixed fluid sample prior to application of the buffered, mixed fluid sample to the solid phase membrane, the particles pass over the capture zone(s) in a continuous manner through the capillary action of the fluid, rather than in a quick wave on the crest of a fluid front. As a result, a lower concentration of particles flows through the capture zone(s) for a longer time: thus the time during which particles can be “captured” is effectively increased, allowing higher specific binding at the capture zones while the amount of particles that pass through the capture zone(s) is effectively lowered, thereby avoiding the non-specific, physical blocking of capture of some particles by others which occurs when the particles pass on the crest of a fluid front. Furthermore, an assessment can be made for multiple analytes, using a single internal control, thereby facilitating analysis of several compounds concurrently. In addition, the assay conditions at the sample capture zone and the control capture zone are as similar as possible: because they are equidistant from the application point, the flow rate at each capture zone will be extremely similar. Also, because the sample capture zone and the control capture zone are encountered by the liquid without any previous encounter with any capture zone, any possible interference attributed to flow through a first sample capture zone into a second subsequent capture zone is eliminated.

Although the assays of the invention have been described particularly in relation to immunoassays, the assays can similarly be used with other binding pairs as described above (e.g., nucleic acids, receptor-ligands, lectin-sugars), using the same methods as described above with the desired components as the analyte and the and the analyte binding agent.

Kits of the Invention

The invention also includes kits for use in the methods described herein. Kit components can include: first and/or second members of a specific binding pair, buffers and/or buffer containers, fluid collection means, one or more solid phase apparatus (optionally comprising an application pad and/or wicking pad), at least one sample collection apparatus, one or more buffer containers, control samples for generation of a standard curve and/or other standard curve information, analyte binding particles, analyte coated particles, and/or control particles, capture reagents, antibodies, tools to assist in collecting of samples to be assessed for analyte of interest (e.g., swabs), disposal apparatus (e.g., biohazard waste bags), and/or other information or instructions regarding the sample collection apparatus (e.g., lot information, expiration date, etc.). For example, in one embodiment, a kit comprises at least one sample collection apparatus having analyte binding particles within it; in a preferred embodiment, a kit comprises at least one pipette tip having evaporatively-dried, vacuum-dried or freeze-dried analyte binding particles therein. In another embodiment, a kit comprises at least one solid phase apparatus as described herein and at least one sample collection apparatus. In another preferred embodiment, a kit comprises at least one pipette; at least one or more pipette tips having evaporatively-dried, vacuum-dried or freeze-dried analyte binding particles therein; and at least one solid phase apparatus. This preferred embodiment can also optionally contain information regarding the standard curve, lot information, and/or expiration date relating to the analyte binding particles in the pipette tips. In yet another preferred embodiment, a kit comprises at least one sample collection apparatus; at least one pipette tip having dried analyte binding particles thereon; at least one solid phase apparatus; and at least one buffer container. This preferred embodiment can also optionally contain buffer within the buffer container; and tool (e.g., a swab) for collection of a solid sample.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for quantitatively measuring the amount of an analyte of interest in a fluid sample, comprising:
a) providing a solid phase apparatus comprising a membrane comprising an application point, a sample capture zone, and a control capture zone; the sample capture zone having a sample capture reagent adsorbed thereon and the control capture zone having a control capture reagent adsorbed thereon; wherein the sample capture zone and the control capture zone are approximately equidistant from the application point;
b) providing a sample collection apparatus containing a population of analyte binding particles, wherein the analyte binding particles are coated with an analyte binding agent;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus,
thereby producing a buffered, mixed fluid sample comprising contacted analyte binding particles;
d) applying the buffered, mixed fluid sample to the application point of the membrane;
e) maintaining the membrane under conditions which allow fluid to transport contacted analyte binding particles by capillary action through the strip to and through the sample capture zone, thereby allowing contacted analyte binding particles to bind to the sample capture reagent; and concurrently allowing the fluid in the sample to transport contacted analyte binding particles by capillary action through the strip to and through the control capture zone, thereby allowing contacted analyte binding particles to bind to the control capture reagent;
f) further maintaining the membrane under conditions which allow the fluid in the sample to transport any contacted analyte binding particles not bound to the sample capture reagent or to the control capture reagent by capillary action beyond the sample capture zone or beyond the control capture zone;
g) determining the amount of contacted analyte binding particles in the sample capture zone and the amount of contacted analyte binding particles in the control capture zone;
h) determining a corrected analyte binding particle amount from the amount of analyte binding particles in the sample capture zone and the amount of analyte binding particles in the control capture zone,
wherein the amount of analyte of interest in the fluid sample is directly related to the corrected analyte binding particle amount.

2. The method of claim 1, wherein the corrected analyte binding particle amount is determined as a ratio of the amount of analyte binding particles in the sample capture zone, to the amount of analyte binding particles in the control capture zone.

3. The method of claim 1, wherein the corrected analyte binding particle amount is determined as a ratio of the amount of analyte binding particles in the sample capture zone, to the sum of the amount of analyte binding particles in the control capture zone and the amount of analyte binding particles in the sample capture zone.

4. The method of claim 1, wherein the analyte and the analyte binding agent are members of a binding pair, and one member of the binding pair is selected from the group consisting of: a spore, a protein, a hormone, an enzyme, a glycoprotein, a peptide, a small molecule, a polysaccharide, a lectin, an antibody, an antibody fragment, a nucleic acid, a drug, a drug conjugate, a toxin, a virus, a virus particle, a portion of a cell wall, a hapten, and a receptor.

5. The method of claim 1, wherein the analyte binding agent is selected from the group consisting of: an antibody; an antibody fragment; a hapten; a drug conjugate; and a receptor.

6. The method of claim 5, wherein the analyte binding agent is an antibody.

7. The method of claim 6, wherein the sample capture reagent is an antibody selected from the group consisting of: an antibody directed against the same epitope as the antibody on the analyte binding particles, and an antibody directed against a different epitope as the antibody on the analyte binding particles.

8. The method of claim 5, wherein the control capture reagent is an anti-immunoglobulin antibody.

9. The method of claim 1, wherein the fluid sample is selected from the group consisting of: whole blood, plasma, serum, urine, cerebrospinal fluid, saliva, semen, vitreous fluid, and synovial fluid.

10. The method of claim 1, wherein the fluid sample comprises a suspended solid.

11. The method of claim 10, wherein the solid is selected from the group consisting of: a particulate sample, a powder sample, a soil sample, and spores.

12. The method of claim 1, wherein the fluid sample is selected from the group consisting of: water, groundwater, sewage, and wastewater.

13. The method of claim 1, wherein in step (d) the mixed fluid sample is applied to the application point through an application pad.

14. The method of claim 1, wherein in step (f) the fluid in the sample transports any contacted analyte binding particles not bound to the sample capture reagent or to the control capture reagent by capillary action beyond the sample capture zone or the control capture zone into a wicking pad.

15. The method of claim 1, wherein the sample collection apparatus is selected from the group consisting of: a pipette and a pipette tip.

16. The method of claim 1, wherein the population of analyte binding particles are evaporatively-dried, vacuum-dried or freeze-dried.

17. The method of claim 1, wherein the sample capture zone and the control capture zone are radially dispersed around the application point.

18. The method of claim 1, wherein the sample capture zone and the control capture zone are adjacent to one another and situated in parallel along the direction of fluid flow by capillary action.

19. A method for quantitatively measuring the amount of an analyte of interest in a fluid sample, comprising:
a) providing a solid phase apparatus comprising a membrane comprising an application point, a sample capture zone, and a control capture zone; the sample capture zone having a sample capture reagent adsorbed thereon and the control capture zone having a control capture reagent adsorbed thereon; wherein the sample capture zone and the control capture zone are approximately equidistant from the application point;
b) providing a sample collection apparatus containing a population of analyte coated particles, wherein the analyte coated particles are coated with analyte or an analog of the analyte;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus, thereby producing a buffered, mixed fluid sample comprising analyte coated particles;
d) applying the buffered, mixed fluid sample to the application point of the membrane;
e) maintaining the membrane under conditions which allow fluid to transport analyte coated particles by capillary action through the strip to and through the sample capture zone, thereby allowing analyte coated particles to bind to the sample capture reagent; and concurrently allowing the fluid in the sample to transport analyte coated particles by capillary action through the strip to and through the control capture zone, thereby allowing analyte coated particles to bind to the control capture reagent;
f) further maintaining the membrane under conditions which allow the fluid in the sample to transport any analyte coated particles not bound to the sample capture reagent or to the control capture reagent by capillary action beyond the sample capture zone or beyond the control capture zone;
g) determining the amount of analyte coated particles in the sample capture zone and the amount of analyte coated particles in the control capture zone;
h) determining a corrected analyte coated particle amount from the amount of analyte coated particles in the sample capture zone and the amount of analyte coated particles in the control capture zone,
wherein the amount of analyte of interest in the fluid sample is inversely related to the corrected analyte coated particle amount.

20. The method of claim 19, wherein the corrected analyte coated particle amount is determined as a ratio of the amount of analyte coated particles in the sample capture zone, to the amount of analyte coated particles in the control capture zone.

21. The method of claim 19, wherein the corrected analyte coated particle amount is determined as a ratio of the amount of analyte coated particles in the sample capture zone, to the sum of the amount of analyte coated particles in the control capture zone and the amount of analyte coated particles in the sample capture zone.

22. The method of claim 19, wherein the analyte and the sample capture reagent are members of a binding pair, and one member of the binding pair is selected from the group consisting of: a spore, a protein, a hormone, an enzyme, a glycoprotein, a peptide, a small molecule, a polysaccharide, a lectin, an antibody, an antibody fragment, a nucleic acid, a drug, a drug conjugate, a toxin, a virus, a virus particle, a portion of a cell wall, a hapten, and a receptor.

23. The method of claim 19, wherein the sample capture reagent is selected from the group consisting of: an antibody; an antibody fragment; a hapten; a drug conjugate; and a receptor.

24. The method of claim 23, wherein the sample capture reagent is an antibody.

25. The method of claim 23, wherein the control capture reagent is an anti-immunoglobulin antibody.

26. The method of claim 19, wherein the fluid sample is selected from the group consisting of: whole blood, plasma, serum, urine, cerebrospinal fluid, saliva, semen, vitreous fluid, and synovial fluid.

27. The method of claim 19, wherein the fluid sample comprises a suspended solid.

28. The method of claim 27, wherein the solid is selected from the group consisting of: a particulate sample, a powder sample, a soil sample, and spores.

29. The method of claim 19, wherein the fluid sample is selected from the group consisting of: water, groundwater, sewage, and wastewater.

30. The method of claim 19, wherein in step (d) the mixed fluid sample is applied to the application point through an application pad.

31. The method of claim 19, wherein in step (f) the fluid in the sample transports any analyte coated particles not bound to the sample capture reagent or to the control capture reagent by capillary action beyond the control capture zone or the control capture zone into a wicking pad.

32. The method of claim 19, wherein the sample collection apparatus is selected from the group consisting of: a pipette and a pipette tip.

33. The method of claim 19, wherein the population of analyte coated particles are evaporatively-dried, vacuum-dried or freeze-dried.

34. The method of claim 19, wherein the sample capture zone and the control capture zone are radially dispersed around the application point.

35. The method of claim 19, wherein the sample capture zone and the control capture zone are adjacent to one another and situated in parallel along the direction of fluid flow by capillary action.

36. A method for quantitatively measuring the amount of at least two analytes of interest in a fluid sample, comprising:
a) providing a solid phase apparatus comprising a membrane comprising an application point, at least two sample capture zones, and a control capture zone; the first sample capture zone having a first sample capture reagent adsorbed thereon, the second sample capture zone having a second sample capture reagent adsorbed thereon, and the control capture zone having a control capture reagent adsorbed thereon; wherein each sample capture zone and the control capture zone are approximately equidistant from the application point;
b) providing a sample collection apparatus containing a population of first analyte binding particles and a population of second analyte binding particles, wherein the first analyte binding particles are coated with a first analyte binding agent and the second analyte binding particles are coated with a second analyte binding agent;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus, thereby producing a buffered, mixed fluid sample comprising contacted first analyte binding particles and contacted second analyte binding particles;
d) applying the buffered, mixed fluid sample to the application point of the membrane;
e) maintaining the membrane under conditions which allow fluid to transport contacted first analyte binding particles and contacted second analyte binding particles by capillary action through the strip to and through each sample capture zone, thereby allowing contacted first analyte binding particles to bind to the first sample capture reagent in the first sample capture zone, and allowing contacted second analyte binding particles to bind to the second sample capture reagent in the second sample capture zone; and concurrently allowing the fluid in the sample to transport contacted first analyte binding particles and contacted second analyte binding particles by capillary action through the strip to and through the control capture zone, thereby allowing contacted first analyte binding particles and contacted second analyte binding particles to bind to the control capture reagent;
f) further maintaining the membrane under conditions which allow the fluid in the sample to transport any contacted first analyte binding particles and contacted second analyte binding particles not bound to a sample capture reagent or to the control capture reagent by capillary action beyond the sample capture zones or beyond the control capture zone;
g) determining the amount of contacted first analyte binding particles in the first sample capture zone, the amount of contacted second analyte binding particles in the second capture zone, and the amount of contacted first analyte binding particles and contacted second analyte binding particles in the control capture zone;
h) determining a first corrected analyte binding particle amount from the amount of contacted first analyte binding particles in the first sample capture zone and the amount of contacted first analyte binding particles and contacted second analyte binding particles in the control capture zone, and a second corrected analyte binding particle amount from the amount of contacted second analyte binding particles in the second sample capture zone and the amount of contacted first analyte binding particles and contacted second analyte binding particles in the control capture zone,
wherein the amount of the first analyte of interest in the fluid sample is directly related to the first corrected analyte binding particle amount, and the amount of the second analyte of interest in the fluid sample is directly related to the second corrected analyte binding particle amount.

37. The method of claim 36, wherein the sample capture zones and the control capture zone are radially dispersed around the application point.

38. The method of claim 36, wherein the sample capture zones and the control capture zone are adjacent to one another and situated in parallel along the direction of fluid flow by capillary action.

39. The method of claim 36, further comprising quantitatively measuring the amount of one or more additional analytes of interest, wherein the membrane comprises an additional sample capture zone for each additional analyte of interest, each additional sample capture zone having a sample capture reagent adsorbed thereon; wherein a sample collection apparatus further contains a population of additional analyte binding particles for each additional analyte of interest; wherein the membrane is maintained under conditions which allow fluid to transport contacted additional analyte binding particles by capillary action through the strip to and through each sample capture zone, thereby allowing contacted additional analyte binding particles to bind to the additional sample capture reagent in each additional sample capture zone; wherein a corrected analyte binding particle amount is determined for each analyte of interest from the amount of contacted additional analyte binding particles in each corresponding additional sample capture zone and the amount of all analyte binding particles in the control capture zone, and wherein the amount of each analyte of interest in the fluid sample is directly related to a corresponding corrected analyte binding particle amount.

40. The method of claim 36, wherein the two analytes of interest are the same analyte.

41. The method of claim 40, wherein the corrected analyte binding particle amounts are averaged and the amount of analyte of interest is related to average corrected analyte binding particle amount.

42. A method for quantitatively measuring the amount of at least two analytes of interest in a fluid sample, comprising: p1 a) providing a solid phase apparatus comprising a membrane comprising an application point, at least two sample capture zones, and a control capture zone; the first sample capture zone having a first sample capture reagent adsorbed thereon, the second sample capture zone having a second sample capture reagent adsorbed thereon, and the control capture zone having a control capture reagent adsorbed thereon; wherein each sample capture zone and the control capture zone are approximately equidistant from the application point;
b) providing a sample collection apparatus containing a population of analyte binding particles, wherein the analyte binding particles are coated with a first analyte binding agent and a second analyte binding agent;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus, thereby producing a buffered, mixed fluid sample comprising contacted analyte binding particles;
d) applying the buffered, mixed fluid sample to the application point of the membrane;
e) maintaining the membrane under conditions which allow fluid to transport contacted analyte binding particles by capillary action through the strip to and through each sample capture zone, thereby allowing contacted analyte binding particles to bind to the first sample capture reagent in the first sample capture zone, and allowing contacted analyte binding particles to bind to the second sample capture reagent in the second sample capture zone; and concurrently allowing the fluid in the sample to transport contacted analyte binding particles by capillary action through the strip to and through the control capture zone, thereby allowing contacted analyte binding particles to bind to the control capture reagent;
f) further maintaining the membrane under conditions which allow the fluid in the sample to transport any contacted analyte binding particles not bound to a sample capture reagent or to the control capture reagent by capillary action beyond the sample capture zones or beyond the control capture zone;
g) determining the amount of contacted analyte binding particles in the first sample capture zone, the amount of contacted analyte binding particles in the second capture zone, and the amount of contacted analyte binding particles in the control capture zone;
h) determining a first corrected analyte binding particle amount from the amount of contacted analyte binding particles in the first sample capture zone and the amount of contacted analyte binding particles in the control capture zone, and a second corrected analyte binding particle amount from the amount of contacted analyte binding particles in the second sample capture zone and the amount of contacted analyte binding particles in the control capture zone,wherein the amount of the first analyte of interest in the fluid sample is directly related to the first corrected analyte binding particle amount, and the amount of the second analyte of interest in the fluid sample is directly related to the second corrected analyte binding particle amount.

43. The method of claim 42, wherein the sample capture zones and the control capture zone are radially dispersed around the application point.

44. The method of claim 42, wherein the sample capture zones and the control capture zone are adjacent to one another and situated in parallel along the direction of fluid flow by capillary action.

45. The method of claim 42, further comprising quantitatively measuring the amount of one or more additional analytes of interest, wherein the membrane comprises an additional sample capture zone for each additional analyte of interest, each additional sample capture zone having a sample capture reagent adsorbed thereon; wherein the analyte binding particles in the sample collection apparatus further comprise additional analyte binding agent coated on the analyte binding particles for each additional analyte of interest; wherein the membrane is maintained under conditions which allow fluid to transport additional contacted analyte binding particles by capillary action through the strip to and through each sample capture zone, thereby allowing contacted analyte binding particles to bind to the additional sample capture reagent in each additional sample capture zone; wherein a corrected analyte binding particle amount is determined for each analyte of interest from the amount of contacted analyte binding particles in each corresponding additional sample capture zone and the amount of contacted analyte binding particles in the control capture zone, and wherein the amount of each analyte of interest in the fluid sample is directly related to a corresponding corrected analyte binding particle amount.

46. The method of claim 42, wherein the two analytes of interest are the same analyte.

47. The method of claim 46, wherein the corrected analyte binding particle amounts are averaged and the amount of analyte of interest is related to average corrected analyte binding particle amount.

48. A method for quantitatively measuring the amount of at least two analytes of interest in a fluid sample, comprising:
a) providing a solid phase apparatus comprising a membrane comprising an application point, at least two sample capture zones, and a control capture zone; the first sample capture zone having a first sample capture reagent adsorbed thereon, the second sample capture zone having a second sample capture reagent adsorbed thereon, and the control capture zone having a control capture reagent adsorbed thereon; wherein each sample capture zone and the control capture zone are approximately equidistant from the application point;
b) providing a sample collection apparatus containing a population of first analyte coated particles and a population of second analyte coated particles, wherein the first analyte coated particles are coated with first analyte or an analog of the first analyte, and the second analyte coated particles are coated with second analyte or an analog of the second analyte;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus,
thereby producing a buffered, mixed fluid sample comprising first analyte coated particles and second analyte coated particles;
d) applying the buffered, mixed fluid sample to the application point of the membrane;
e) maintaining the membrane under conditions which allow fluid to transport first analyte coated particles and second analyte coated particles by capillary action through the strip to and through each sample capture zone, thereby allowing first analyte coated particles to bind to the first sample capture reagent in the first sample capture zone, and allowing second analyte coated particles to bind to the second sample capture reagent in the second sample capture zone; and concurrently allowing the fluid in the sample to transport first analyte coated particles and second analyte coated by capillary action through the strip to and through the control capture zone, thereby allowing first analyte coated particles and second analyte coated particles to bind to the control capture reagent;
f) further maintaining the membrane under conditions which allow the fluid in the sample to transport any first analyte coated particles and second analyte coated particles not bound to a sample capture reagent or to the control capture reagent by capillary action beyond the sample captures zone or beyond the control capture zone;
g) determining the amount of first analyte coated particles in the first sample capture zone, the amount of second analyte coated particles in the second sample capture zone, and the amount of first analyte coated particles and second analyte coated particles in the control capture zone;
h) determining a first corrected analyte coated particle amount from the amount of first analyte coated particles in the first sample capture zone and the amount of first analyte coated particles and second analyte coated particles in the control capture zone, and a second corrected analyte coated particle amount from the amount of second analyte coated particles in the second sample capture zone and the amount of first analyte coated particles and second analyte coated particles in the control capture zone,
wherein the amount of the first analyte of interest in the fluid sample is inversely related to the first corrected analyte coated particle amount, and the amount of the second analyte of interest in the fluid sample is inversely related to the second corrected analyte coated particle amount.

49. The method of claim 48, wherein the sample capture zones and the control capture zone are radially dispersed around the application point.

50. The method of claim 48, wherein the sample capture zones and the control capture zone are adjacent to one another and situated in parallel along the direction of fluid flow by capillary action.

51. The method of claim 48, further comprising quantitatively measuring the amount of one or more additional analytes of interest, wherein the membrane comprises an additional sample capture zone for each additional analyte of interest, each additional sample capture zone having a sample capture reagent adsorbed thereon; wherein a sample collection apparatus further contains a population of additional analyte coated particles for each additional analyte of interest; wherein the membrane is maintained under conditions which allow fluid to transport additional analyte coated particles by capillary action through the strip to and through each sample capture zone, thereby allowing additional analyte coated particles to bind to the additional sample capture reagent in each additional sample capture zone; wherein a corrected analyte binding particle amount is determined for each analyte of interest from the amount of additional analyte coated particles in each corresponding additional sample capture zone and the amount of analyte coated particles in the control capture zone, and wherein the amount of each analyte of interest in the fluid sample is directly related to a corresponding corrected analyte coated particle amount.

52. The method of claim 48, wherein the two analytes of interest are the same analyte.

53. The method of claim 52, wherein the corrected analyte coated particle amounts are averaged and the amount of analyte of interest is related to average corrected analyte binding particle amount.

54. A method for quantitatively measuring the amount of at least two analytes of interest in a fluid sample, comprising:
a) providing a solid phase apparatus comprising a membrane comprising an application point, at least two sample capture zones, and a control capture zone; the first sample capture zone having a first sample capture reagent adsorbed thereon, the second sample capture zone having a second sample capture reagent adsorbed thereon, and the control capture zone having a control capture reagent adsorbed thereon; wherein each sample capture zone and the control capture zone are approximately equidistant from the application point;
b) providing a sample collection apparatus containing a population of analyte coated particles, wherein the analyte coated particles are coated with first analyte or an analog of the first analyte, and with second analyte or an analog of the second analyte;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus,
thereby producing a buffered, mixed fluid sample comprising analyte coated particles;
d) applying the buffered, mixed fluid sample to the application point of the membrane;
e) maintaining the membrane under conditions which allow fluid to transport analyte coated particles by capillary action through the strip to and through each sample capture zone, thereby allowing analyte coated particles to bind to the first sample capture reagent in the first sample capture zone, and allowing analyte coated particles to bind to the second sample capture reagent in the second sample capture zone; and concurrently allowing the fluid in the sample to transport analyte coated particles by capillary action through the strip to and through the control capture zone, thereby allowing analyte coated particles to bind to the control capture reagent;
f) further maintaining the membrane under conditions which allow the fluid in the sample to transport any analyte coated particles not bound to a sample capture reagent or to the control capture reagent by capillary action beyond the sample captures zone or beyond the control capture zone;
g) determining the amount of analyte coated particles in the first sample capture zone, the amount of analyte coated particles in the second sample capture zone, and the amount of analyte coated particles in the control capture zone;
h) determining a first corrected analyte coated particle amount from the amount of analyte coated particles in the first sample capture zone and the amount of analyte coated particles in the control capture zone, and a second corrected analyte coated particle amount from the amount of analyte coated particles in the second sample capture zone and the amount of analyte coated particles in the control capture zone,
wherein the amount of the first analyte of interest in the fluid sample is inversely related to the first corrected analyte coated particle amount, and the amount of the second analyte of interest in the fluid sample is inversely related to the second corrected analyte coated particle amount.

55. The method of claim 54, wherein the sample capture zones and the control capture zone are radially dispersed around the application point.

56. The method of claim 54, wherein the sample capture zones and the control capture zone are adjacent to one another and situated in parallel along the direction of fluid flow by capillary action.

57. The method of claim 54, further comprising quantitatively measuring the amount of one or more additional analytes of interest, wherein the membrane comprises an additional sample capture zone for each additional analyte of interest, each additional sample capture zone having a sample capture reagent adsorbed thereon; wherein the population of analyte coated particles further comprises each additional analyte of interest coated on the particles; wherein the membrane is maintained under conditions which allow fluid to transport additional contacted analyte coated particles by capillary action through the strip to and through each sample capture zone, thereby allowing contacted additional analyte coated particles to bind to the additional sample capture reagent in each additional sample capture zone; wherein a corrected analyte binding particle amount is determined for each analyte of interest from the amount of additional analyte coated particles in each corresponding additional sample capture zone and the amount of analyte coated particles in the control capture zone, and wherein the amount of each analyte of interest in the fluid sample is directly related to a corresponding corrected analyte coated particle amount.

58. The method of claim 54, wherein the two analytes of interest are the same analyte.

59. The method of claim 58, wherein the corrected analyte coated particle amounts are averaged and the amount of analyte of interest is related to average corrected analyte binding particle amount.

60. A method for quantitatively measuring the amount of multiple analytes of interest in a fluid sample, comprising:
a) providing a solid phase apparatus comprising a membrane comprising an application point, a sample capture zone for each analyte of interest, and a control capture zone; each sample capture zone having a sample capture reagent that is a binding partner of an analyte of interest adsorbed thereon, and the control capture zone having a control capture reagent adsorbed thereon; wherein each sample capture zone and the control capture zone are approximately equidistant from the application point;
b) providing a sample collection apparatus containing populations of analyte binding particles, wherein each population of analyte binding particles is coated with an analyte binding agent for an analyte of interest, such that one population of analyte binding particles is present for each analyte of interest;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus, thereby producing a buffered, mixed fluid sample comprising contacted analyte binding particles;
d) applying the buffered, mixed fluid sample to the application point of the membrane;
e) maintaining the membrane under conditions which allow fluid to transport contacted analyte binding particles by capillary action through the strip to and through each sample capture zone, thereby allowing contacted analyte binding particles to bind to the sample capture reagent in the sample capture zone for each analyte of interest; and concurrently allowing the fluid in the sample to transport contacted analyte binding particles by capillary action through the strip to and through the control capture zone, thereby allowing contacted analyte binding particles to bind to the control capture reagent;
f) further maintaining the membrane under conditions which allow the fluid in the sample to transport any contacted analyte binding particles not bound to a sample capture reagent or to the control capture reagent by capillary action beyond the sample capture zones or beyond the control capture zone;
g) determining the amount of contacted analyte binding particles in each sample capture zone, and the amount of contacted analyte binding particles in the control capture zone;
h) determining a corrected analyte binding particle amount for each analyte of interest, from the amount of analyte binding particles in the corresponding sample capture zone and the amount of analyte binding particles in the control capture zone, wherein the amount of an analyte of interest in the fluid sample is directly related to the corrected analyte binding particle amount.

61. The method of claim 60, wherein the sample capture zones and the control capture zone are radially dispersed around the application point.

62. The method of claim 60, wherein the sample capture zones and the control capture zone are adjacent to one another and situated in parallel along the direction of fluid flow by capillary action.

63. A method for quantitatively measuring the amount of multiple analytes of interest in a fluid sample, comprising:
a) providing a solid phase apparatus comprising a membrane comprising an application point, a sample capture zone for each analyte of interest, and a control capture zone; each sample capture zone having a sample capture reagent that is a binding partner of an analyte of interest adsorbed thereon, and the control capture zone having a control capture reagent adsorbed thereon; wherein each sample capture zone and the control capture zone are approximately equidistant from the application point;
b) providing a sample collection apparatus a population of analyte binding particles, wherein the population of analyte binding particles is coated with an analyte binding agent for each analyte of interest, such that analyte binding agents are present for each analyte of interest;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus, thereby producing a buffered, mixed fluid sample comprising contacted analyte binding particles;
d) applying the buffered, mixed fluid sample to the application point of the membrane;
e) maintaining the membrane under conditions which allow fluid to transport contacted analyte binding particles by capillary action through the strip to and through each sample capture zone, thereby allowing contacted analyte binding particles to bind to the sample capture reagent in the sample capture zone for each analyte of interest; and concurrently allowing the fluid in the sample to transport contacted analyte binding particles by capillary action through the strip to and through the control capture zone, thereby allowing contacted analyte binding particles to bind to the control capture reagent;
f) further maintaining the membrane under conditions which allow the fluid in the sample to transport any contacted analyte binding particles not bound to a sample capture reagent or to the control capture reagent by capillary action beyond the sample capture zones or beyond the control capture zone;
g) determining the amount of contacted analyte binding particles in each sample capture zone, and the amount of contacted analyte binding particles in the control capture zone;
h) determining a corrected analyte binding particle amount for each analyte of interest, from the amount of analyte binding particles in the corresponding sample capture zone and the amount of analyte binding particles in the control capture zone,
wherein the amount of an analyte of interest in the fluid sample is directly related to the corrected analyte binding particle amount.

64. The method of claim 63, wherein the sample capture zones and the control capture zone are radially dispersed around the application point.

65. The method of claim 63, wherein the sample capture zones and the control capture zone are adjacent to one another and situated in parallel along the direction of fluid flow by capillary action.

66. A method for quantitatively measuring the amount of multiple analytes of interest in a fluid sample, comprising:
a) providing a solid phase apparatus comprising a membrane comprising an application point, a sample capture zone for each analyte of interest, and a control capture zone; each sample capture zone having a sample capture reagent that is a binding partner of an analyte of interest adsorbed thereon, and the control capture zone having a control capture reagent adsorbed thereon; wherein each sample capture zone and the control capture zone are approximately equidistant from the application point;
b) providing a sample collection apparatus containing populations of analyte coated particles, wherein each population of analyte coated particles is coated with an analyte of interest or an analog of an analyte of interest, such that one population of analyte coated particles is present for each analyte of interest;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus, thereby producing a buffered, mixed fluid sample comprising analyte coated particles;
d) applying the buffered, mixed fluid sample to the application point of the membrane;
e) maintaining the membrane under conditions which allow fluid to transport analyte coated particles by capillary action through the strip to and through each sample capture zone, thereby allowing analyte coated particles to bind to the sample capture reagent in the sample capture zone for each analyte of interest; and concurrently allowing the fluid in the sample to transport analyte coated particles by capillary action through the strip to and through the control capture zone, thereby allowing analyte coated particles to bind to the control capture reagent;
f) further maintaining the membrane under conditions which allow the fluid in the sample to transport any analyte coated particles not bound to a sample capture reagent or to the control capture reagent by capillary action beyond the sample capture zones or beyond the control capture zone;
g) determining the amount of analyte coated particles in each sample capture zone, and the amount of analyte coated particles in the control capture zone;
h) determining a corrected analyte coated particle amount for each analyte of interest, from the amount of analyte coated particles in the corresponding sample capture zone and the amount of analyte coated particles in the control capture zone,
wherein the amount of an analyte of interest in the fluid sample is inversely related to the corrected analyte coated particle amount.

67. The method of claim 66, wherein the sample capture zones and the control capture zone are radially dispersed around the application point.

68. The method of claim 66, wherein the sample capture zones and the control capture zone are adjacent to one another and situated in parallel along the direction of fluid flow by capillary action.

69. A method for quantitatively measuring the amount of multiple analytes of interest in a fluid sample, comprising:
a) providing a solid phase apparatus comprising a membrane comprising an application point, a sample capture zone for each analyte of interest, and a control capture zone; each sample capture zone having a sample capture reagent that is a binding partner of an analyte of interest adsorbed thereon, and the control capture zone having a control capture reagent adsorbed thereon; wherein each sample capture zone and the control capture zone are approximately equidistant from the application point;
b) providing a sample collection apparatus a population of analyte coated particles, wherein the population of analyte coated particles is coated with analyte of interest or an analog of an analyte of interest, for each analyte of interest, such that analyte or analog of analyte are present for each analyte of interest;
c) either i) introducing the fluid sample into the sample collection apparatus, producing a mixed fluid sample, and subsequently introducing a buffer into the mixed fluid sample; ii) introducing a buffer into the sample collection apparatus and subsequently introducing the fluid sample; or iii) forming the fluid sample by introducing a solid into a buffer, and subsequently introducing the fluid sample into the sample collection apparatus, thereby producing a buffered, mixed fluid sample comprising analyte coated particles;
d) applying the buffered, mixed fluid sample to the application point of the membrane;
e) maintaining the membrane under conditions which allow fluid to transport analyte coated particles by capillary action through the strip to and through each sample capture zone, thereby allowing analyte coated particles to bind to the sample capture reagent in the sample capture zone for each analyte of interest; and concurrently allowing the fluid in the sample to transport analyte coated particles by capillary action through the strip to and through the control capture zone, thereby allowing analyte coated particles to bind to the control capture reagent;
f) further maintaining the membrane under conditions which allow the fluid in the sample to transport any analyte coated particles not bound to a sample capture reagent or to the control capture reagent by capillary action beyond the sample capture zones or beyond the control capture zone;
g) determining the amount of analyte coated particles in each sample capture zone, and the amount of analyte coated particles in the control capture zone;
h) determining a corrected analyte coated particle amount for each analyte of interest, from the amount of analyte coated particles in the corresponding sample capture zone and the amount of analyte coated particles in the control capture zone,
wherein the amount of an analyte of interest in the fluid sample is inversely related to the corrected analyte binding particle amount.

70. The method of claim 69, wherein the sample capture zones and the control capture zone are radially dispersed around the application point.

71. The method of claim 69, wherein the sample capture zones and the control capture zone are adjacent to one another and situated in parallel along the direction of fluid flow by capillary action.
